(19) United States
(12) Patent Application Publication
(10) Pub. No.: US 2010/0055076 A1
(43) Pub. Date: Mar. 4, 2010

(54) Mesenchymal stem cell-mediated autologous dendritic cells with increased immunosuppression

(75) Inventors: Dae Seog Lim, Goyang-si (KR); Ju Ah Jeong, Seoul (KR); Mi-Sun Kang, Seoul (KR); Hyun Soo Lee, Seoul (KR); Yong Soo Bae, Suwon-si (KR)

Correspondence Address: THE NATH LAW GROUP, 112 South West Street, Alexandria, VA, 22314, US

(73) Assignee: CREAGENE INC., Sungnam-si (KR)

(21) Appl. No.: 12/461,694

(22) Filed: Aug. 20, 2009

Related U.S. Application Data
(63) Continuation-in-part of application No. PCT/KR2007/003681, filed on Jul. 31, 2007.

(30) Foreign Application Priority Data
Feb. 22, 2007 (KR) 10-2007-0017970

(51) Int. Cl. A61K 35/12; C12N 5/00; C12N 5/02

(52) U.S. Cl. 424/93.7; 435/325; 435/373

(57) ABSTRACT

A method for preparing dendritic cells which have an enhanced potential to suppress immune responses, method for suppressing immune response by comprising administering them, dendritic cells carrying a potential to suppress immune responses, and a pharmaceutical composition comprising the dendritic cells capable of inducing immunosuppressive responses. The present dendritic cells having an enhanced potential to suppress immune responses can be utilized for treating or preventing various diseases or disorders through the suppression of immune responses. In addition, the enhanced immunotolerance potential of the dendritic cells ensures the cells to be effectively used as an immunosuppressive agent.

### CROSS-REFERENCE TO RELATED APPLICATIONS

This application is a continuation-in-part of PCT Application No. PCT/KR2007/003681, filed Jul. 31, 2007, which claims priority to Korean Patent Application No. 10-2007-0017970, filed on Feb. 22, 2007, the contents of which are both hereby expressly incorporated by reference in their entirety.

### FIELD OF THE INVENTION

The present subject matter relates to mesenchymal stem cell-mediated autologous dendritic cells having an enhanced potential to suppress immune responses, preparing method thereof, method for suppressing immune responses by comprising administering them and pharmaceutical compositions comprising them.

### BACKGROUND OF THE INVENTION

Mesenchymal stem cells (MSCs) are adult progenitor cells present in the bone marrow (Bm) that are able to differentiate into several lineages, such as adipocytes, osteoblasts, and chondrocytes (1). MSCs have been isolated from a number of species, including human (1), mouse (2), rat (3), canine (4), goat, rabbit (5) and feline (6). Murine MSCs are far more difficult to be isolated from the bone marrow and expanded in culture than human or rat MSCs (7). In contrast to human and rat MSCs, the cultures of murine MSCs are frequently contaminated by hematopoietic progenitors that outgrow the cultures. MSCs have been recently demonstrated to suppress several T-lymphocyte activities, thus exerting an immunoregulatory capacity both in vitro and in vivo (8, 9). MSCs significantly prolong the survival of MHC-mismatched skin grafts after infusion in baboons and reduce the incidence of graft-versus-host disease (GVHD) after allogeneic hematopoietic stem cell (HSC) transplantation in humans (8, 10). However, the mechanisms involved in the immunoregulatory activity of MSCs on T lymphocytes are still partially obscure, and side effects of stem cells themselves in vivo also remain unclear.

Dendritic cells (DCs) are known as established inducers of T-cell immunity and are also increasingly viewed as mediators of T-cell tolerance (11, 12). In contrast to mature DCs (mDCs), the nature function of imDCs is to provide conditions for self-tolerance, either through the generation of T_{reg} cells, or through induction of apoptosis or anergy of autoreactive effector cells (13-15). Several attempts have been made to utilize imDCs therapeutically. Unfortunately, some obstacles including limited generation protocols and the occurrence of a maturation event in the host, still exist that prevent the therapeutic use of imDCs (16, 17). Nevertheless, it is obvious through some reports that imDCs have a tolerogenic feature activating T_{reg} cells or inducing anergy of effector T cells (18, 19).

In mice, both imDCs and mDCs can maintain the expansion of CD25⁺ CD4⁺ T_{reg} cells (20), although mDCs can also inhibit CD25⁺ CD4⁺ T_{reg} cell-mediated immune suppression through the production of IL-6 (21). DC expression of CD40 is an important factor determining whether priming will result in immunity or T_{reg}-mediated immune suppression. Antigen-exposed DCs which lack CD40 prevent T cell priming, suppress previously primed immune responses and induce IL-10-secreting CD4⁺ T_{reg} cells that can transfer antigen-specific tolerance to primed recipients (22).

Throughout this application, various patents and publications are referenced and citations are provided in parentheses. The disclosure of these patents and publications in their entirety are hereby incorporated by references into this application in order to more fully describe the present subject matter and the state of the art to which this subject matter pertains.

### SUMMARY OF THE INVENTION

The present subject matter arose as the result of intensive research to prepare cells for immunotherapy which exert immunosuppressive activity and do not possess a tendency to generate tumors at the same time. As a result, the present application relates to the discovery that where dendritic cells are co-cultured with mesenchymal stem cells, the potential of the dendritic cells isolated from the co-culture media to suppress immune responses is significantly enhanced.

Accordingly, it is an object of this subject matter to provide dendritic cells having an enhanced potential to suppress immune responses.

It is another object of this subject matter to provide dendritic cells which are mediated by mesenchymal stem cells.

It is still another object of this subject matter to provide a pharmaceutical composition comprising dendritic cells which are mediated by mesenchymal stem cells.

It is another object of this subject matter to provide methods for suppressing immune responses comprising administering to a subject a pharmaceutically effective amount of dendritic cells mediated by mesenchymal stem cells.

Other objects and advantages of the present subject matter will become apparent from the following detailed description together with the appended claims and drawings.

In one aspect of this subject matter, there is provided a method for preparing dendritic cells, which comprises the steps of: (a) preparing dendritic cells; (b) preparing mesenchymal stem cells; (c) co-culturing the dendritic cells with the mesenchymal stem cells; and (d) isolating dendritic cells having an enhanced potential to suppress immune responses from the co-cultured medium.

In another aspect of this subject matter, there is provided a mesenchymal stem cell-mediated dendritic cell for suppressing immune responses.

According to a preferred embodiment, the present mesenchymal stem cell-mediated dendritic cell is co-cultured with a mesenchymal stem cell so that it has an enhanced ability to suppress immune-active T cells and to induce the regulatory T cells.

According to another preferred embodiment, the present mesenchymal stem cell-mediated dendritic cell is co-cultured with mesenchymal stem cell so that it has a potential to suppress the secretion of inflammatory cytokines and to promote the secretion of immunosuppressive cytokines.

### BRIEF DESCRIPTION OF THE DRAWINGS

FIG. 1 shows results of flow cytometry analysis of mesenchymal stem cells (MSCs) derived from bone marrow, and confirmation of pluripotency of MSCs. FIG. 1A shows results of flow cytometry analysis of MSCs by using cell surface markers. FIG. 1B is a photograph of isolated MSCs. FIG. 1C, FIG. 1D and FIG. 1E show the result that MSCs have been differentiated into adipocytes, osteoblasts and chondrocytes respectively.

FIGS. 2A to 2D show results of FACS analysis of the immature dendritic cells treated with MSCs using typical DC markers.

FIGS. 3A to 3F show results of co-culture of splenocytes with mesenchymal stem cells and/or immature dendritic cells and/or mature dendritic cells. The proportion of Foxp3⁺ T_{reg} cell population derived from splenocytes was analyzed by flow cytometry. Foxp3⁺ T_{reg} cell population was greatly induced from splenocytes mediated by co-culturing with MSCs and imDCs for 72 hr, as compared with splenocytes treated by co-culturing with other cell combinations.

FIG. 4 shows results of the TGF-β expression level where splenocytes were co-cultured with MSCs and imDCs. FIG. 4A displays the TGF-β secretion induced from the co-culture of imDCs, MSCs and splenocytes. FIG. 4B exhibits the TGF-β secretion induced from the co-culture of imDCs, MSCs and CD4+. FIG. 4C shows RT-PCR analysis of TGF-β transcript expressed in imDCs from the co-culture of imDCs and MSCs for 72 hr. The expression of TGF-β, which acts as an immune suppression agent, was highly induced in imDCs from the co-culture of imDCs and MSCs for 72 hr compared to the culture of imDCs for 72 hr (see Lane 5).

FIG. 5 shows the secretion of IFN-γ (Th1 cytokines), IL-4 and IL-10 (Th2 cytokines) in the co-culture of CD4⁺ T cells with MSCs and/or imDCs. FIG. 5A shows results that the secretion of IFN-γ was dramatically increased in the co-culture of CD4⁺ T cells with imDCs, on the contrary, the secretion of IFN-γ was significantly reduced in the co-culture of CD4⁺ T cells with imDCs and MSCs. FIG. 5B reveals results that the secretion of IL-4 was increased in the co-culture of CD4⁺ T cells with imDCs and MSCs compared to the co-culture of CD4⁺ T cells with MSCs (similar level with the co-culture of CD4⁺ T cells with imDCs). FIG. 5C displays results that the secretion of IL-10 was significantly induced in the co-culture of CD4⁺ T cells with MSCs and imDCs.

FIG. 6 shows IL-10 secretion rate of imDCs, mDCs, and MSCs-mediated imDCs respectively.

FIG. 7a-7d shows results with regard to the tumor growth in mice allografted with B16 melanoma cells in the presence or absence of immunosuppressive cells. FIG. 7a shows results that in all tested groups except for imDC-injected group and control group tumor incidence was 100% during the first 11 days. FIG. 7b shows photograph indicating B16 tumor-injected Balb/c mice generating tumor. The first photograph displays the mouse without transplantation of immunosuppressive cells and the second and third photo-images represent mice injected with the MSC-mediated imDCs. FIG. 7c reveals the distribution of CD25⁺ Foxp3⁺ T_{reg} cell population in the CD4⁺ T cells isolated from each mouse group transplanted with immunosuppressive cells and B16 melanoma cells. The mouse injected with MSC-mediated DCs had the largest CD25⁺ Foxp3⁺ T_{reg} cell population. FIG. 7d shows TGF-β concentrations in the serum of the mouse injected with immunosuppressive cells and B16 melanoma cells respectively. It can be understood that the concentration of TGF-β in the group of the mouse injected with MSC-mediated DCs was slightly higher than other groups.

FIG. 8 shows that survival rates of Balb/c mice grafted with B16 melanoma cells co-injected with immune cells of MSCs, imDCs, MSCs+imDCs or MSC-mediated imDCs respectively. It is clearly understood that the co-injected MSC-mediated imDCs increased the survival of Balb/c mice grafted with B16 melanoma compared with other immune cells.

### DETAILED DESCRIPTION OF THE INVENTION

The present subject matter arose as the result of intensive research to prepare cells for immunotherapy which exert immunosuppressive activity and do not possess a tendency to generate tumors at the same time. As a result, the present application relates to the discovery that where dendritic cells are co-cultured with mesenchymal stem cells, the potential of dendritic cells to suppress immune responses is significantly enhanced.

Accordingly, the present method will be explained without restraint in the followings.

(a) Preparation of Dendritic Cells

According to the present subject matter, the potential of dendritic cells to suppress immune responses can be remarkably enhanced by treating dendritic cells derived from mammals, preferably from humans, with mesenchymal stem cells.

The term “dendritic cells (DCs)” used herein refers to antigen-presenting cells, which are capable of presenting antigen to T cells through MHC (major histocompatibility complex). DCs are classified into immature dendritic cells and mature dendritic cells according to the extent of maturity.

The term “immature dendritic cells (imDCs)” used herein refers to a population of dendritic cells which are differentiated from various precursors and show low expressing levels of the surface phenotypes of mature DCs such as costimulatory molecules of CD80 or CD86.

The term “mature dendritic cells (mDCs)” used herein refers to a population of dendritic cells which are matured from imDCs and express at least one of surface phenotypes such as reduced expression of CD115, CD14 or CD68; and increased expression of CD11c, CD80, CD86, CD40, MHC class II, p55 and CD83.

The expression profiling of these surface markers is able to be carried out by the flow cytometry analysis known to those skilled in the art.

The dendritic cells of the instant subject matter are preferably mature or immature dendritic cells, more preferably immature dendritic cells.

General procedures for isolating and culturing immature DCs are disclosed in U.S. Pat. No. 5,994,126 and WO 97/29182, which are incorporated herein by references in their entirety.

Suitable source for isolating immature dendritic cells is tissue that contains immature dendritic cells or their progenitors, and specifically include spleen, afferent lymph, bone marrow, blood, and cord blood, as well as blood cells elicited after administration of cytokines such as G-CSF or FLT-3 ligand.

According to a specific embodiment of this subject matter, a tissue source may be treated prior to culturing with substances that stimulate hematopoiesis, such as, for example, G-CSF, FLT-3, GM-CSF, M-CSF, TGF-β, and thrombopoietin in order to increase the proportion of dendritic cell precursors relative to other cell types.

Such pretreatment may also remove cells which may compete with the proliferation of the dendritic cell precursors or inhibit their survival. Pretreatment may also be used to make the tissue source more suitable for in vitro culture. Those skilled in the art would recognize that the method of treatment will likely depend on the particular tissue source. For example, spleen or bone marrow would first be treated so as to obtain single cells followed by suitable cell separation techniques to separate leukocytes from other cell types as described in U.S. Pat. Nos. 5,851,756 and 5,994,126 which are herein incorporated by reference in their entirety. Treatment of blood would preferably involve cell separation techniques to separate leukocytes from other cell types including red blood cells (RBCs) which are toxic. Removal of RBCs may be accomplished by standard methods known in the art. According to a preferred embodiment of the present subject matter, the tissue source is blood or bone marrow.

According to a further embodiment, immature dendritic cells are derived from multipotent blood monocyte precursors (see WO 97/29182). These multipotent cells typically express CD14, CD32, CD68 and CD115 monocyte markers with little or no expression of CD83, or p55 or accessory molecules such as CD40 and CD86. When cultured in the presence of cytokines such as a combination of GM-CSF and IL-4 or IL-13 as described below, the multipotent cells give rise to the immature dendritic cells. The immature dendritic cells can be modified, for example using vectors expressing IL-10 to keep them in an immature state in vitro or in vivo.

Those skilled in the art would recognize that any number of modifications may be introduced to the disclosed methods for isolating immature dendritic cells and maintaining them in an immature state in vitro and in vivo having regard to the objects of the several embodiments of the subject matter here disclosed.

Cells obtained from the appropriate tissue source are cultured to form a primary culture, preferably, on an appropriate substrate in a culture medium supplemented with granulocyte/macrophage colony-stimulating factor (GM-CSF), a substance which promotes the differentiation of pluripotent cells to immature dendritic cells as described in U.S. Pat. Nos. 5,851,756 and 5,994,126, which are herein incorporated by reference in their entirety. In a preferred embodiment, the substrate would include any tissue compatible surface to which cells may adhere. Preferably, the substrate is commercial plastic treated for use in tissue culture.

To further increase the yield of immature dendritic cells, other factors, in addition to GM-CSF, may be added to the culture medium which block or inhibit proliferation of non-dendritic cell types. Examples of factors which inhibit non-dendritic cell proliferation include interleukin-4 (IL-4) and/or interleukin-13 (IL-13), which are known to inhibit macrophage proliferation. The combination of these substances increases the number of immature dendritic cells present in the culture by preferentially stimulating proliferation of the dendritic cell precursors, while at the same time inhibiting growth of non-dendritic cell types.

According to a specific example herein, an enriched population of immature dendritic cells can be generated from blood monocyte precursors by plating mononuclear cells on plastic tissue culture plates and allowing them to adhere. The plastic adherent cells are then cultured in the presence of GM-CSF and IL-4 in order to expand the population of immature dendritic cells. Other cytokines such as IL-13 may be employed instead of using IL-4.

A medium useful in the procedure of obtaining immature dendritic cells includes any conventional medium for culturing animal cells, preferably, a medium containing serum (e.g., fetal bovine serum, horse serum and human serum). The medium used herein includes, but is not limited to, for example, RPMI series (e.g., RPMI 1640), Eagles's MEM (Eagle's minimum essential medium, Eagle, H. Science 130:432 (1959)), A-MEM (Stanner, C. P. et al., Nat. New Biol. 230:52 (1971)), Iscove's MEM (Iscove, N. et al., J. Exp. Med. 147:923 (1978)), 199 medium (Morgan et al., Proc. Soc. Exp. Bio. Med. 73:1 (1950)), CMRL 1066, RPMI 1640 (Moore et al., J. Amer. Med. Assoc. 199:519 (1967)), F12 (Ham, Proc. Natl. Acad. Sci. USA 53:288 (1965)), F10 (Ham, R.G. Exp. Cell Res. 29:515 (1963)), DMEM (Dulbecco's modification of Eagle's medium, Dulbecco, R. et al., Virology 8:396 (1959)), Mixture of DMEM and F12 (Barnes, D. et al., Anal. Biochem. 102:255 (1980)), Way-mouth's MB752/1 (Waymouth, C. J. Natl. Cancer Inst. 22:1003 (1959)), McCoy's 5A (McCoy, T. A., et al., Proc. Soc. Exp. Biol. Med. 100:115 (1959)) and MCDB series (Ham, R. G. et al., In Vitro 14:11 (1978)) but not limited to. The medium may contain other components, for example, antioxidant (e.g., β-mercaptoethanol). The detailed description of media is found in R. Ian Freshney, Culture of Animal Cells, A Manual of Basic Technique, Alan R. Liss, Inc., New York, the teaching of which is incorporated herein by reference in its entity.

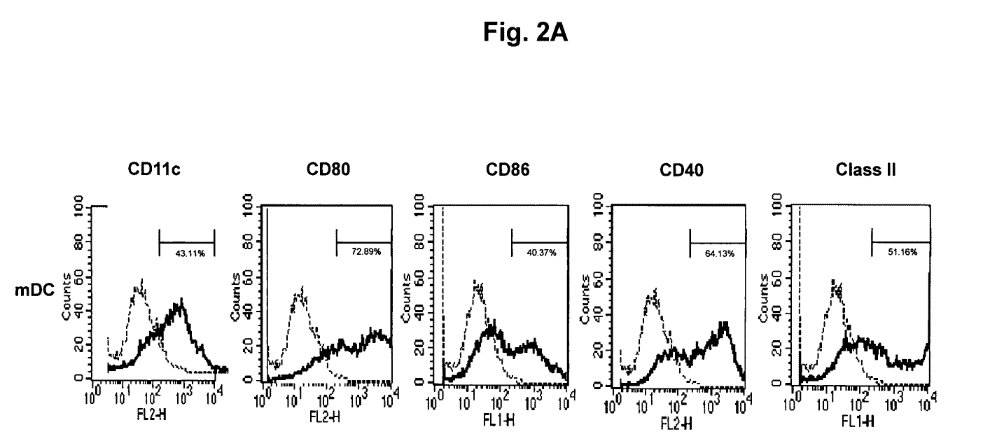

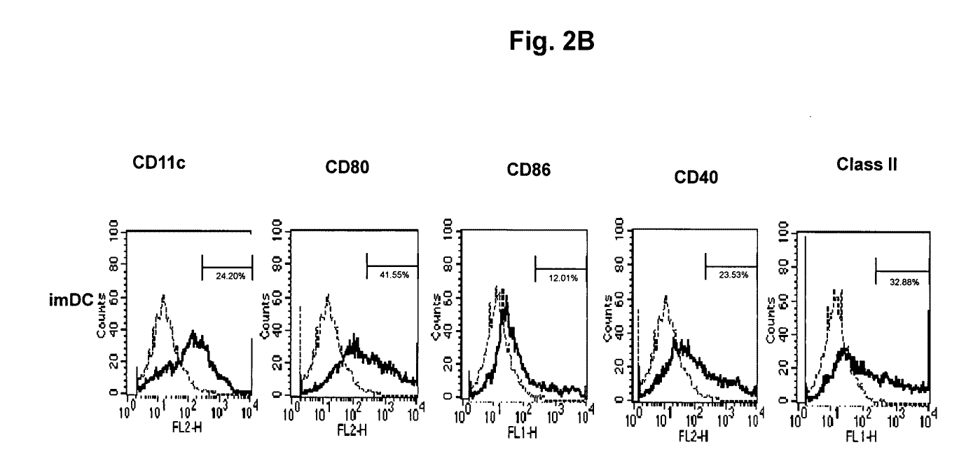

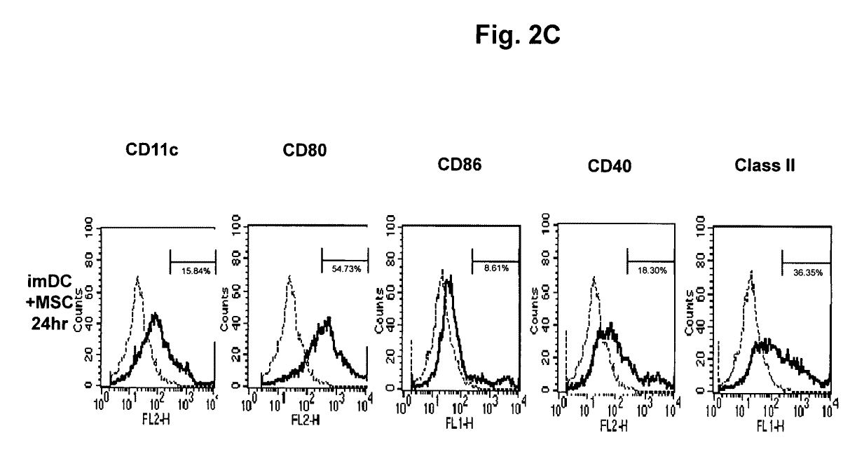

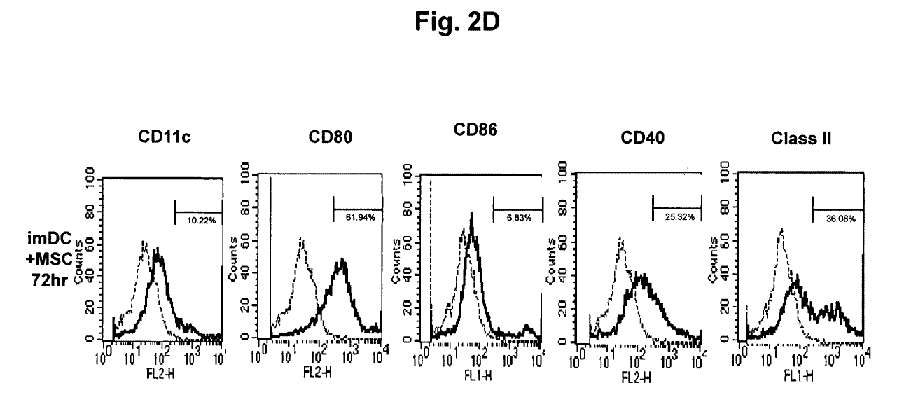

Examples of markers for mature dendritic cells include, for example, expression of surface CD83, DC-LAMP, p55, CCR-7, and high expression level of MHC II and costimulatory molecule such as CD86 (see FIGS. 2A to 2D). Immature dendritic cells are identified based on typical morphology, expression of lower levels of MHC II and costimulatory molecules (see FIGS. 2A to 2D), and the lack of expression of DC maturation markers, e.g., surface expression of CD83 and expression of DC-LAMP. In addition, examples of positive markers for immature dendritic cells include, but are not limited to, DC-SIGN, Langerin and CD1A.

Thus, by utilizing standard antibody staining techniques known in the art, it is possible to assess the proportion of immature dendritic cells in any given culture. Antibodies may also be used to isolate or purify immature dendritic cells from mixed cell cultures by flow cytometry or other cell sorting techniques well known in the art.

(b) Preparation of Mesenchymal Stem Cells (MSCs)

According to a method of the present subject matter, dendritic cells are co-cultured with mesenchymal stem cells in order to enhance their potential to suppress immune responses.

The term “mesenchymal stem cells (MSCs)” used herein refers to the pluripotential cells found inter alia in bone marrow, blood, dermis and periosteum that are capable of differentiating into any of the specific types of mesenchymal or connective tissues (i.e. the tissues of the body that support the specialized elements; particularly adipose, osseous, cartilaginous, elastic, and fibrous connective tissues) depending upon various influences from bioactive factors, such as cytokines.

The mesenchymal stem cells of this subject matter may be derived from animals, preferably from mammals, more preferably from humans. According to a specific example, the mesenchymal stem cells derived from a mouse are used.

The mesenchymal stem cells are present in bone marrow in very minute amounts and the general procedures for isolating and culturing mesenchymal stem cells are described in U.S. Pat. No. 5,486,359 which is herein incorporated by reference in its entirety. Mesenchymal stem cells can be isolated from tissue and purified when cultured in a specific medium by their selective attachment, termed “adherence” to substrates.

The procedures for isolating, purifying and culturing mesenchymal stem cells are explained as follows according to a specific example.

Mesenchymal stem cells are isolated from mammals including human and mouse, preferably from a human source such as blood or bone marrow. The bone marrow may be extracted from tibias, femurs, spinal cord, ilium. The cells obtained from bone marrow are cultured in a suitable medium. Removing floating cells and sub-culturing adherent cells results in established mesenchymal stem cells.

A medium useful in the procedure of preparing mesenchymal stem cells includes any conventional medium for culturing stem cells, preferably, a medium containing serum (e.g., fetal bovine serum, horse serum and human serum).

The medium used herein includes, but is not limited to, for example, RPMI series (e.g., RPMI 1640), Eagles's MEM (Eagle's minimum essential medium, Eagle, H. Science 130:432 (1959)), α-MEM (Stanner, C. P. et al., Nat. New Biol. 230:52 (1971)), Iscove's MEM (Iscove, N. et al. J. Exp. Med. 147:923 (1978)), 199 medium (Morgan et al., Proc. Soc. Exp. Bio. Med., 73:1 (1950)), CMRL 1066, RPMI 1640 (Moore et al., 3. Amer. Med. Assoc. 199:519 (1967)), F12 (Ham, Proc. Natl. Acad. Sci. USA 53:288 (1965)), F10 (Ham, R.G. Exp. Cell Res. 29:515 (1963)), DMEM (Dulbecco's modification of Eagle's medium, Dulbecco, R. et al., Virology 8:396 (1959)), a mixture of DMEM and F12 (Barnes, D. et al., Anal. Biochem. 102:255 (1980)), Way-mouth's MB752/1 (Waymouth, C. J. Natl. Cancer Inst. 22:1003 (1959)), McCoy's 5A (McCoy, T. A., et al., Proc. Soc. Exp. Biol. Med. 100:115 (1959)) and MCDB series (Ham, R. G. et al., In Vitro 14:11 (1978)).

The medium may contain other components, for example, antibiotics or antifungal agent (e.g., penicillin, streptomycin) and glutamine. The detailed description of media is found in R. Ian Freshney, Culture of Animal Cells, A Manual of Basic Technique, Alan R. Liss, Inc., New York, the teaching of which is incorporated herein by reference in its entity.

The mesenchymal stem cells can be identified by using flow cytometry which may be carried out with specific surface markers of MSCs. For example, mesenchymal stem cells are positive for CD44, CD29 and MHC class I.

According to a preferred embodiment of this subject matter, mesenchymal stem cells utilized herein are positive for surface markers of CD44, CD29 and MHC class I and are negative for CD14, CD45, CD54, MHC class II and CD11b. The term “positive” used herein with reference to the stem cells and surface markers means an aspect in which the antibodies to the surface markers of the stem cells specifically binds to markers where the stem cells are treated with the antibodies.

The mesenchymal stem cells isolated and established through the above-mentioned procedures have an ability to proliferate without differentiation, and capable of being differentiated into various types of cell where the cells are induced to differentiate.

(c) Co-culture of Dendritic Cells with Mensenchymal Stem Cells; and (d) Isolation of Dendritic Cells Having an Enhanced Potential to Suppress Immune Responses from the Co-Culture.

According to the method of this subject matter, the isolated dendritic cells and mesenchymal stem cells are co-cultured. Co-culturing may be carried out according to the conventional methods for culturing animal cells. A medium useful in the procedure of co-culturing includes any conventional medium for animal cells culture, preferably, a medium containing serum (e.g., fetal bovine serum, horse serum and human serum).

The medium used herein includes, but is not limited to, for example, RPMI series (e.g., RPMI 1640), Eagles's MEM (Eagle's minimum essential medium, Eagle, H. Science 130:432 (1959)), α-MEM (Stanner, C. P. et al., Nat. New Biol. 230:52 (1971)), Iscove's MEM (Iscove, N. et al., J. Exp. Med. 147:923 (1978)), 199 medium (Morgan et al., Proc. Soc. Exp. Bio. Med., 73:1 (1950)), CMRL 1066, RPMI 1640 (Moore et al., J. Amer. Med. Assoc. 199:519 (1967)), F12 (Ham, Proc. Natl. Acad. Sci. USA 53:288 (1965)), F10 (Ham, R.G. Exp. Cell Res. 29:515 (1963)), DMEM (Dulbecco's modification of Eagle's medium, Dulbecco, R. et al., Virology 8:396 (1959)), a mixture of DMEM and F12 (Barnes, D. et al., Anal. Biochem. 102:255 (1980)), Way-mouth's MB752/1 (Waymouth, C. J. Natl. Cancer Inst. 22:1003 (1959)), McCoy's 5A (McCoy, T. A., et al., Proc. Soc. Exp. Biol. Med. 100:115 (1959)) and MCDB series (Ham, R. G. et al., In Vitro 14:11 (1978)).

The detailed description of media is found in R. Ian Freshney, Culture of Animal Cells, A Manual of Basic Technique, Alan R. Liss, Inc., New York, the teaching of which is incorporated herein by reference in its entity.

The dendritic cells in the co-culture step of the present methods are syngeneic, allogeneic or xenogeneic to the mesenchymal stem cells. Preferably, the dendritic cells are syngeneic or allogeneic to the mesenchymal stem cells.

The co-culture of dendritic cells with mesenchymal stem cells is carried out for a period of time for dendritic cells to obtain an enhanced potential to suppress immune responses and the co-culture time is not limited to a specific one, preferably 0.1-200 hr, more preferably 1-100 hr, still more preferably 10-90 hr, most preferably 30-80 hr.

Where dendritic cells are co-cultured with mesenchymal stem cells, the ratio of the number of dendritic cells to mesenchymal stem cells is not specifically limited. The ratio of the number of mesenchymal stem cells to the number of dendritic cells is 1000:1-1:1000, more preferably 500:1-1:500, still more preferably 100:1-1:100, most preferably 10:1-1:20.

According to a preferred embodiment of this subject matter, no cytokine is added into the media for co-culture of immature dendritic cells and mesenchymal stem cells. The cytokines that are not added into the co-culture media includes, but are not limited to, for example GM-CSF, TNF-α, IL-3, and IL-4.

According to another preferred embodiment of the instant subject matter, any dendritic cells maturation stimulating factor is not added into the media for co-culture of immature dendritic cells and mesenchymal stem cells. The dendritic cells maturation stimulating factor includes, but is not limited to, for example lipopolysaccharide and CD40L.

Since mesenchymal stem cells are adherent cells and dendritic cells are non-adherent cells, the dendritic cells having an enhanced potential to suppress immune responses can be obtained by isolating the floating cells from the co-cultured medium.

According to a preferred embodiment of this subject matter, the dendritic cells finally obtained according to the present method and having an enhanced potential to suppress immune responses possess an increased expression level of CD80 compared to the dendritic cells in the step (a).

According to a preferred embodiment of this subject matter, the dendritic cells finally obtained according to the present method and having an enhanced potential to suppress immune responses carry increased expression levels of MHC II class compared to the dendritic cells in the step (a).

According to a preferred embodiment of this subject matter, the dendritic cells finally obtained according to the present method and having an enhanced potential to suppress immune responses have reduced expression levels of CD86 compared to the dendritic cells in the step (a).

According to a preferred embodiment of this subject matter, the dendritic cells finally obtained according to the present method and having an enhanced potential to suppress immune responses possess increased expression levels of CD11c compared to the dendritic cells in the step (a).

According to a preferred embodiment of this subject matter, the dendritic cells finally obtained according to the present method and having an enhanced potential to suppress immune response have increased potential to secrete IL(Interleukin)-10 compared to the dendritic cells in the step(a).

The method of this subject matter makes it possible to effectively prepare dendritic cells having a remarkably enhanced potential to suppress immune responses with high reproducibility.

The immature dendritic cells having an enhanced potential to suppress immune responses are also referred to as “mesenchymal stem cell-mediated dendritic cells” herein.

The term used herein “mediated” refers to contacting dendritic cells with mesenchymal stem cells, and preferably refers to preparation of the dendritic cells having an enhanced potential to suppress immune responses by co-culturing them with mesenchymal stem cells.

Thus, the expression “mesenchymal stem cell-treated dendritic cells” are used interchangeably herein with the term “mesenchymal stem cell-mediated dendritic cells.”

The dedritic cells of the present subject matter obtained by co-culturing with mesenchymal stem cells exert significantly enhanced activities to suppress immune responses.

The immune tolerance induced by the mesenchymal stem cell-mediated dendritic cells is the result of immunosuppressive effect exerted by CD25⁺ Foxp3⁺ specific T_{reg} cells. T_{reg} cells have been reported to suppress the activities, proliferation, differentiation and effector function of the various types of immune cell including CD4⁺ and CD8⁺ T cells, B cells, NK cells and dendritic cells (25). Although the mechanism of immune suppression induced by T_{reg} cell has not been exactly elucidated, it is well known that T_{reg} cell exerts its immunosuppressive effect through the induction of immunosuppressive cytokines such as TGF-β and IL-10, or the cell to cell interactions mediated by suppressive receptor CTLA-4 (26, 27).

The immature dendritic cells of the instant subject matter significantly increase the population of CD25⁺ Foxp3⁺ T_{reg} cells which exhibit immunosuppressive activities and remarkably enhance the secretion of immunosuppressive cytokine TGF-β. In addition, the dendritic cells of this subject matter suppress the secretion of IFN-γ (Th1 cytokine) and promote the secretion of IL-4 and IL-10 (Th2 cytokine), and as a result decrease the ratio of Th1/Th2.

In another aspect of this subject matter, there is provided a pharmaceutical composition for suppressing immune responses, which comprises (a) a pharmaceutically effective amount of mesenchymal stem cell-mediated dendritic cells; and (b) a pharmaceutically acceptable carrier.

In another aspect of this subject matter, there is provided a method for suppressing immune responses, which comprise administering to a subject in need for immune suppression a pharmaceutically effective amount of mesenchymal stem cell-mediated dendritic cells.

Considering the side effects of stem cells that likely generate tumors when injected into a subject, administration of the mesenchymal stem cell-mediated (treated) dendritic cells of this subject matter has great advantages of expecting a potential to suppress immune responses equal or superior to that of stem cells without the dangers of tumorigenesis.

The term used herein “for suppressing immune responses” means a use to suppress immune responses in the recipient. Thus, the pharmaceutical composition of this subject matter can be used to administer to a recipient in need of immune suppression in order to effectively suppress immune responses. The present composition can be used to treat various diseases or disorders.

The term used herein “subject” or “recipient” is meant to include an animal, preferably mammals such as human and mouse, most preferably human, which is suffering from immune diseases or has the dangers of tissue or organ transplantation rejection.

The present pharmaceutical composition includes mesenchymal stem cell-mediated dendritic cells having an enhanced potential to suppress immune responses as an active ingredient. Since the present composition comprises, in principle, the dendritic cells described above, the common descriptions between them are omitted in order to avoid undue redundancy leading to the complexity of this specification.

According to a preferred embodiment, autologous or syngeneic dendritic cells are used in co-culture with mesenchymal stem cells. Most preferably autologous dendritic cells are employed in the present subject matter. Since a pharmaceutical composition of this invention contains autologous immature dendritic cells which have been derived from a subject, it has advantages of little elicitation of immune responses to the injected dendritic cells.

Disorders or diseases that may be treated or prevented by administering the compositions of the subject matter include any one which can be treated or prevented by suppressing immune responses. Thus, disorders or diseases that can be treated or prevented by the present composition include the autoimmune disorders, inflammatory diseases and graft rejection.

Examples of autoimmune disorders that may be treated or prevented by the present pharmaceutical compositions include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre syndrome, Hashimoto's thyroiditis, idiopathic pulmonary fibrosis, idiopathic thrombocytopenia purpura (ITP), irritable bowel disease (IBD), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, stiff-man syndrome, systemic lupus erythematosus, lupus erythematosus, takayasu arteritis, temporal arteristis, giant cell arteritis, ulcerative colitis, uveitis, vitiligo and Wegener's granulomatosis.

Preferably autoimmune disorders that may be treated or prevented by the present pharmaceutical compositions include, but not limited to rheumatoid arthritis, type 1 diabetes mellitus, multiple sclerosis, systemic lupus erythematosus, and atopy.

Examples of autoimmune disorders that may be treated or prevented by the present pharmaceutical compositions include, but are not limited to, asthma, encephalitis, inflammatory bowel disease, chronic obstructive pulmonary disease (COPD), allergic disorders, pulmonary fibrosis, undifferentiated spondyloarthropathy, undifferentiated arthropathy, arthritis, inflammatory osteolysis, and chronic inflammation resulting from chronic viral or bacteria infections.

The pharmaceutical compositions herein are useful for suppressing graft rejection immune responses in the transplanted tissues, organs or cells. The present compositions are also effective for preventing the transplantation recipient from being aggravated. For example, insulin dependent diabetes mellitus (IDDM), type I diabetes is believed to be an autoimmune disorder resulting from autoimmune responses to β cells in Langerhans islet which secrete insulin. Treating a subject suffering from early state IDDM before his β cells in Langerhans islet being completely destructed is important for preventing further destruction of β cells and the aggravation of diseases.

Based on the standard clinical and laboratory experiments and methods, physicians as an ordinary person skilled in the art can easily select a subject in need of suppressing immune responses.

In the pharmaceutical compositions of this subject matter, the pharmaceutically acceptable carrier may be a conventional one for formulation, including, but not limited to, lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methylhydroxy benzoate, propylhydroxy benzoate, talc, magnesium stearate, and mineral oils. The pharmaceutical composition according to the present invention may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

The pharmaceutical composition according to the present subject matter may be administered via the oral route or parenterally. When the pharmaceutical composition of the present subject matter is administered parenterally, it can be done by intravenous, intraperitoneal, intramuscular, subcutaneous, or local administration. It is desirable that the route of administration of the present composition should be determined according to the disease to which the composition is applied. For example, where the present composition is used to treat or prevent type I diabetes, the intraperitoneal administration is preferable because the administered dendritic cells effectively migrate to pancreas without being diluted. In addition, where the composition of this subject matter is employed to treat or prevent patients suffering from arthritis, it is preferably administered via the intravenous route, most preferably injected into the joint via local administration.

A suitable dose of the pharmaceutical composition of the present subject matter may vary depending on pharmaceutical formulation methods, administration methods, the patient's age, body weight, sex, severity of diseases, diet, administration time, administration route, an excretion rate and sensitivity for a used pharmaceutical composition. Preferably, the pharmaceutical composition of the present subject matter is administered with a daily dose of 1×10³−1×10¹² cells/kg (body weight).

According to the conventional techniques known to those skilled in the art, the pharmaceutical compositions may be formulated with a pharmaceutically acceptable carrier and/or vehicle as described above, finally providing several forms including a unit dose form and a multi-dose form.

In another aspect of this subject matter, there is provided a method for administering into a subject dendritic cells having an enhanced immunosuppressive potential, which have been obtained by co-culturing with immature dendritic cells and mesenchymal stem cells and isolating dendritic cells from the co-cultured medium.

In another aspect of this subject matter, there is provided a method for suppressing immune responses, which comprises administering to a subject a pharmaceutical composition comprising (a) a pharmaceutically effective amount of mesenchymal stem cell-mediated dendritic cells; and (b) a pharmaceutically acceptable carrier.

According to a preferred embodiment, the mesenchymal stem cell-mediated dendritic cells are autologous cells.

According to a preferred embodiment, the mesenchymal stem cell-mediated dendritic cells have reduced CD86 expression level compared to the dendritic cells which are untreated with mesenchymal stem cells.

According to a preferred embodiment, the mesenchymal stem cell-mediated dendritic cells have increased CD80 expression level compared to the dendritic cells which are untreated with mesenchymal stem cells.

According to a preferred embodiment, the mesenchymal stem cell-mediated dendritic cells have a potential to increase the population of CD25⁺ Foxp3⁺ T_{reg} cells.

According to a preferred embodiment, the composition of the instant subject matter is used for treating or preventing tissue or organ transplantation rejection, autoimmune disease, or inflammatory disease.

According to a more preferred embodiment, the composition of the instant subject matter is used for treating or preventing tissue or organ transplantation rejection.

According to a preferred embodiment, the autoimmune disease is rheumatoid arthritis, diabetics, or atopic dermatitis.

The features and advantages of this subject matter can be summarized as follows:

(i) The present subject matter provides a method for preparing dendritic cells having an enhanced potential to suppress immune responses by co-culturing with mesenchymal stem cells and the dendritic cells prepared by this method.

(ii) The instant subject matter provides a pharmaceutical composition for suppressing immune responses, which comprises a pharmaceutically effective amount of mesenchymal stem cell-mediated dendritic cells.

(iii) The present dendritic cells having an enhanced potential to suppress immune responses can be utilized for treating various diseases or disorders through the suppression of immune responses.

(iv) The enhanced immunotolerance capability of the dendritic cells of this invention ensures DCs to be effectively utilized as an immunosuppressive agent.

The present subject matter will now be described in further detail by examples. It would be obvious to those skilled in the art that these examples are intended to be more concretely illustrative and the scope of the present subject matter as set forth in the appended claims is not limited to or by the examples.

### EXAMPLES

### Methods and Materials

Mouse (m) MSC Preparation

Bone marrow from 6-week-old female Balb/c mice (Orient Bio, Gyeonggi-do, Korea) was flushed out of tibias and femurs. After washing by centrifugation (1500 rpm, 3 min) in phosphate-buffered saline (PBS), cells were suspended in cell culture medium comprising LG (low glucose)-DMEM (Life Technologies, Gaithersburg, Md., USA), 15% fetal bovine serum (FBS, RH Biosciences, Lenexa, Kans., USA), 100 U/ml penicillin, 100 μg/ml streptomycin, 2 mM L-glutamine, and 1% antibiotics-antimycotics (Life Technologies, Gaithersburg, Md., USA) and plated in T75 flask. Suspended cells were removed after 5 to 7 days of culture, and adherent cells were continued to culture. Cultures were maintained at 37° C. in a humidified atmosphere containing 5% CO₂ and culture medium was changed every 3 to 4 days. Cells were detached with 0.1% trypsin-EDTA when they reached 50-60% confluence, and replated at a density of 2×10³ cells/cm² in other culture flasks. Homologous adherent cells were characterized by flow cytometric analysis of relevant specific surface markers (see the “FACS Analysis” section). Cells cultured for 4-7 passages were used for further cellular analyses and differentiation experiments.

Differentiation of Bone Marrow-Derived MSCs

To induce adipogenic differentiation, cells were incubated for 2 weeks in adipogenic medium consisting of LG-DMEM supplemented with 0.5 mM 3-isobutyl-1-methylxantine (IBMX), 1 μM hydrocortisone, and 0.1 mM indomethacine (Sigma-Aldrich, St. Louis, Mo., USA). Cell morphology was examined under a phase contrast microscope in order to confirm the formation of neutral lipid vacuoles. The presence of neutral lipids was visualized by staining with oil-red O (Sigma-Aldrich, St. Louis, Mo., USA).

In addition, for osteogenic differentiation, adherent cells were cultured in osteogenic medium consisting of LG-DMEM supplemented with 10% FBS, 10 mM β-glycerophosphate, 100 nM dexamethasone, and 30 μM ascorbate (Sigma-Aldrich, St. Louis, Mo., USA) for 2 weeks. Osteogenic differentiation was evaluated by alkaline phosphatase (ALP) staining. For ALP staining, the mono-layered cells were prefixed with 4% formaldehyde and added with Western blue stabilized substrate (Promega, Madison, Wis., USA) for 30 min at room temperature.

Finally, for chondrogenic differentiation, approximately 5×10⁶ cells in the 15 ml polypropylene tube were centrifuged at 1000 rpm for 5 min to form a pelleted micromass in the bottom of the tube and incubated for up to 5 weeks with chondrogenic medium consisting of LG-DMEM supplemented with 1 mM pyruvate, 0.1 mM ascorbate 2-phosphate, 100 nM dexamethasone, ITS+ premix (6.25 μg/ml insulin, 6.25 μg/ml transferrin, 6.25 μg/ml selenious acid, 5.35 μg/ml linoleic acid, and 1.25 mg/ml bovine serum albumin), 35 nM L-proline and 10 ng/ml recombinant human TGF-β1 (Sigma-Aldrich, St. Louis, Mo., USA). Chondrogenic differentiation was verified by histochemical staining of micromasses with safranin red O (Sigma-Aldrich, St. Louis, Mo., USA).

Generation of Bone Marrow-Derived imDCs

Mouse Bm (bone marrow)-derived imDCs were generated from Balb/c, 6-7 weeks, female mice. After removing all muscle tissues with gauze from the femurs and tibias, the bones were placed in a 60-mm dish with 70% alcohol for a few seconds, washed twice with PBS, and transferred into a fresh dish with RPMI 1640 (Life Technologies, Gaithersburg, Md., USA).

Both ends of the bones were cut with scissors in the dish, and then the marrow was flushed out using 1 ml of RPMI 1640 with a syringe and 26-gauge needle. The tissue was suspended, passed through nylon mesh to remove small pieces of bone and debris, and erythrocytes were lysed with ACK lysing buffer (Cambrex Bio Science Walkersville, Inc., Walkersville, Md., USA).

The Bm cells obtained were cultured at 1×10⁶ cells per a well (in 6-well plate) in RPMI 1640 supplemented with 10% FBS (Gibco BRL, Grand Island, N.Y., USA), 1/1000-diluted 2-mercaptoethanol (Life Technologies, Gaithersburg, Md., USA), 10 ng/ml of mouse recombinant GM-CSF and 10 ng/ml of mouse recombinant IL-4.

The cells were cultured at 37° C. in an atmosphere of 5% CO₂ and 95% humidity. On day 2 the supernatant was removed and replaced with fresh media containing the same supplements. Typical experiments were performed with the nonadherent and loosely adherent cell population from cultures at days 6. In addition, to obtain mDCs, the imDCs were further cultured with 1 μg/ml lipopolysaccharide (LPS, Sigma-Aldrich, St. Louis, Mo., USA) for an additional 24 hr. At the end of the culture period, the cells were characterized by flow cytometric analysis of relevant specific surface markers (see the “FACS analysis” section).

Co-Culture of imDCS and MSCs

To characterize imDCs mediated with MSCs, the cells were plated at a ratio of 1×10⁵ MSCs per 1×10⁶ imDCs and incubated in RPMI 1640 supplemented with 10% FBS for 72 hr. During the co-culture of imDCs and MSCs, any cytokine and DCs maturation stimulating factor was not added into the media for co-culture. After the incubation, suspended cells were analyzed with specific surface markers.

Investigation of T_{Reg} Population and TGF-β Secretion by Mixed Lymphocyte Reaction (MLR)

Splenocytes were isolated from the spleen of Balb/c mice and disaggregated into RPMI 1640 medium. Erythrocytes in them were lysed with ACK lysing buffer for 5 min at room temperature and washed in PBS. Cells prepared (1×10⁶ imDCs and 1×10⁵ MSCs) were co-cultured with 5×10⁶ splenocytes in 6-well plates for 72 hr.

To investigate the change of the T_{reg} population and TGF-β secretion in the MLR cultures, at the end of each culture period (6, 24, 48 and 72 hr), suspended cells in the co-cultures were harvested by centrifugation (1500 rpm, 3 min). The supernatants and pellets were used for TGF-β ELISA and FoxP3 (CD4⁺ CD25⁺ T_{reg}-specific) FACS or TGF-β RT-PCR analysis, respectively. For FoxP3 FACS analysis, CD4 T cells were isolated from the pellets (see below for detailed description). In addition, MSCs and imDCs were also isolated from the co-cultures for RT-PCR analysis

Evaluation of Th1/Th2 Response

Quantitative analysis of Th1 cytokine IFN-γ and Th2 cytokine IL-4 levels was performed by ELISA on supernatants from 24, 42, and 72 hr-MLR cultures using CD4⁺ T cell. CD4⁺ T cells were isolated from splenocytes by use of a CD4 MicroBeads mouse kit (Miltenyi Biotec, Auburn, Calif., USA).

Briefly, CD4 T cells were separated by passing the cell suspension over a magnetic-activated cell sorter MS column held in MACS magnetic separator (Miltenyi Biotec, Auburn, Calif., USA).

The CD4 T cells adhering to the column were then used for this assay. In addition, quantitative analysis of IL-10 levels was performed by ELISA on samples above.

FACS Analysis

For flow cytometric analysis, MSCs were harvested by treatment with 0.1% trypsin-EDTA, and detached cells were washed with PBS and incubated at 4° C. for 30 min with the following cell-specific antibodies; CD11b, CD14, CD29, CD44 (β1 integrin), CD45, major histocompatibility complex (MHC) class I, and MHC class II, all of which were conjugated with either fluorescein isothiocyanate (FITC) or phycoerythrin (PE) (BD biosciences, San Jose, Calif., USA). In addition, the imDCs and MSC-mediated imDCs were washed with PBS after harvest, and labeled with CD11c, CD40, CD80, CD86, and MHC Class II antibodies (BD biosciences, San Jose, Calif., USA). To investigate T_{reg} population, splenocytes or CD4⁺ T cells were cultured with imDCs and/or MSCs and labeled with CD25 and Foxp3 antibodies.

After the labeled cells were washed with PBS, cells were analyzed on a FACS Calibur (BD biosciences, San Jose, Calif., USA) using CellQuest software (BD Biosciences, San Jose, Calif., USA). A total of 10⁴ events for each sample were acquired.

ELISA

TGF-β, IFN-γ, IL-4 and IL-10 concentrations were determined in the MLR culture supernatant using each commercially available kit (R&D systems, Abington, OX, UK) according to the manufacturer's instructions.

RT-PCR

Suspended (imDCs) or adherent (MSCs) cells from the imDC+MSC co-culture were harvested and washed once in cold PBS. Total RNA was extracted using RNeasy Mini isolation kit (Qiagen, Valencia, Calif., USA) according to the provided protocol. The first strand complementary DNA (cDNA) was synthesized using SuperScript™ III First-strand Synthesis System for RT-PCR (Invitrogen, California, Calif., USA). The initial denaturation was performed at 95° C. for 5 min. PCR amplification was carried out at 95° C. for 30 sec, at 57° C. for 30 sec, and 72° C. for 30 sec for a total of 35 cycles and final extension at 72° C. for 7 min using DNA Engine Dyad Peltier Thermal Cycler (MJ Research, Waltham, Mass., USA).

The following sense and antisense primers for each molecule were used for: mTGF-β (187 bp), (sense) 5′-tgcgcttgcagagattaaaa-3′, (antisense) 5′-agccctgtattccgtctcc-3′; (Bionics, Guro, Korea). The PCR products were fractionated by 1% agarose (Promega, Madison, Wis., USA) gel electrophoresis, and the bands were visualized by ethidium bromide (EtBr) staining and photographed with Polaroid 667 (Polaroid Corporation, Waltham, Mass., USA).

Determination of IL-10 Secretion Rate of Dendritic Cells

The following cells were prepared according to the procedure as described in the above: immature dendritic cells (imDCs), mature dendritic cells (mDCs), mesenchymal stem cells-mediated immature dendritic cells (MSCs mediated imDCs; imDCs which were isolated from co-culture media after being co-cultured with MSCs for 72 hr). After the respective cells (1×10⁶ cells) were cultured alone for 2 days, IL-10 secretion rates of the cells were determined using IL-10 ELISA Kit.

Tumor Allograft Assay Using B16 Melanoma Cells

B16F10 melanoma cells, MSCs, imDCs, imDC+MSCs and MSC-mediated imDCs (imDCs after 72 hr co-culture with MSCs) were prepared either as single-cell type suspensions (1×10⁶ cells in 100 μl PBS) or a mix of cells (1×10⁶ imDCs and 1×10⁶ MSCs in 200 μl PBS). Using 7- to 8-week-old Balb/c mice (allogeneic recipients for B16 cells), subcutaneous administration of immune suppressor cells was performed in the left abdominal area.

Instantly after suppressor cell injection, B16 melanoma cells were subcutaneously implanted at a distance of at least 2 cm (the right flank). Mice were examined 3 times a week and tumor growth was evaluated by measuring the length and width of tumor mass (volume=length×width²/2). The tumors were monitored until they reached a volume greater than 30 mm³. The results were presented to be tumor incidence (%, positive: mice bearing tumor mass of more than 30 mm³). At 7 days of the experiments, animals were killed and immune status assays by use of their spleen and serum were performed.

Measurement of Survival Rates of Mice Grafted with B16 Melanoma Cells after Injection of MSCs, imDCs, imDC+MSCs, or MSC-Mediated imDCs.

B16F10 melanoma cells, MSCs, imDCs, imDC+MSCs, and MSC-mediated imDCs (imDCs after 72 hr co-culture with MSCs) were prepared. MSCs, imDCs, imDC+MSCs, and MSC-mediated imDCs were injected respectively into 7- to 8-week-old Balb/c mice (allogeneic recipients for B16 melanoma cells), and after that, B16F10 melanoma cells were subcutaneously implanted. Survival rates of the respective recipient mice were determined for 84 days.

Statistics

Statistical significance (P<0.05) was determined by the two-tailed Student's t test or Mann-Whitney U test.

Results

Characterization of MSCs by Flow Cytometry and Its Multipotentiality

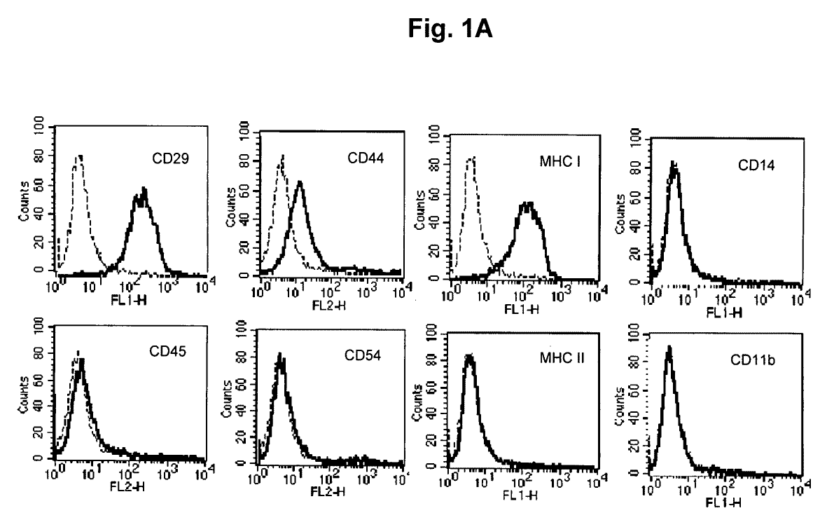

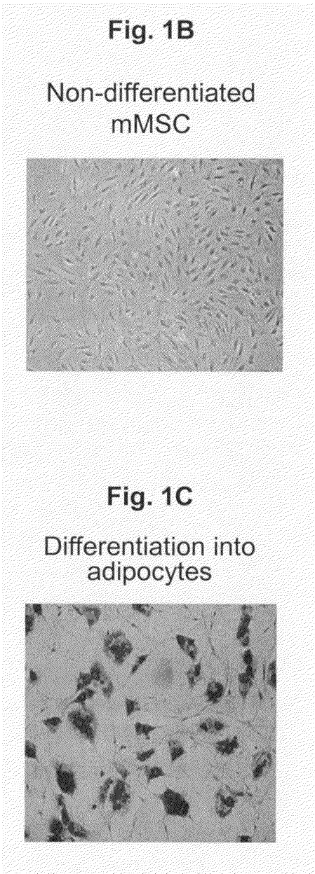

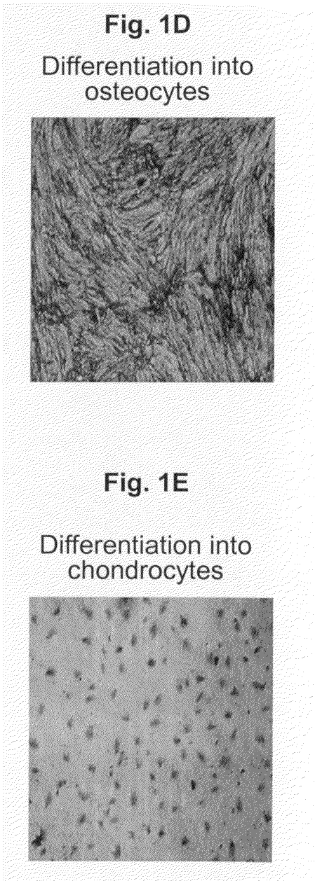

The expression of cell surface antigens was evaluated by flow cytometry on MSCs obtained after four passages in LG-DMEM. These cells failed to mark with haematopoietic markers (CD14, CD45 and CD54) but were positive for the adhesion molecules (CD29 and CD44) and MHC class I. Cells were also negative for a myeloid DC marker CD11b, as well as for MHC class II (FIG. 1A). The phenotype of these cells was identical to the phenotype previously reported for typical MSCs (28, 29).

Three of the MSC cultures were tested for their ability to differentiate into other cell types. When subjected to adipogenic, osteogenic and chondrogenic media, MSCs (FIG. 1 B) clearly differentiated into adipocytes (FIG. 1 C), osteoblasts (FIG. 1 D) and chondrocytes (FIG. 1E), respectively. These data indicate that mMSCs isolated showed multipotentiality for differentiation to other cell types.

MSC-Mediated imDCs Express Typical DC Markers, but Show the Expression of Surface Markers to a Lower Level, as Compared to that of mDCs

We next investigated their phenotypes using typical DC markers by FACS analysis, when imDCs were mediated with MSCs.

As shown in FIGS. 2A to 2D, MSC-mediated imDCs expressed typical DC markers, however showed the expression of their surface markers to a lower level, as compared to that of mDCs, and the expression of surface markers at a similar level as compared to that of imDCs. However, a gradual increase of CD80 (costimulator, B7-1) expression on the surface over time was observed when imDCs were co-cultured with MSCs. Meanwhile, MSC-mediated imDCs showed lower expression of CD86 (B7-2), as compared to that of imDC alone.

The FoxP3⁺ T_{reg} Cell Population was Remarkably Induced from Splenocytes Co-Cultured Along with MSCs and imDCs

To investigate whether the FoxP3⁺ T_{reg} cell population could be induced from splenocytes mediated with MSCs and imDCs, MSCs, imDCs and splenocytes were co-cultured together, and CD4 T cells were then isolated from the co-cultured splenocytes for FACS analysis. FoxP3 (forkhead box P3 transcription factor) is the most specific T_{reg} marker currently available while other molecules (i.e., CD45RB, CD38 and CD62L) previously failed to demonstrate specificity for detecting T_{reg} cells with immunosuppressive activity (25, 26).

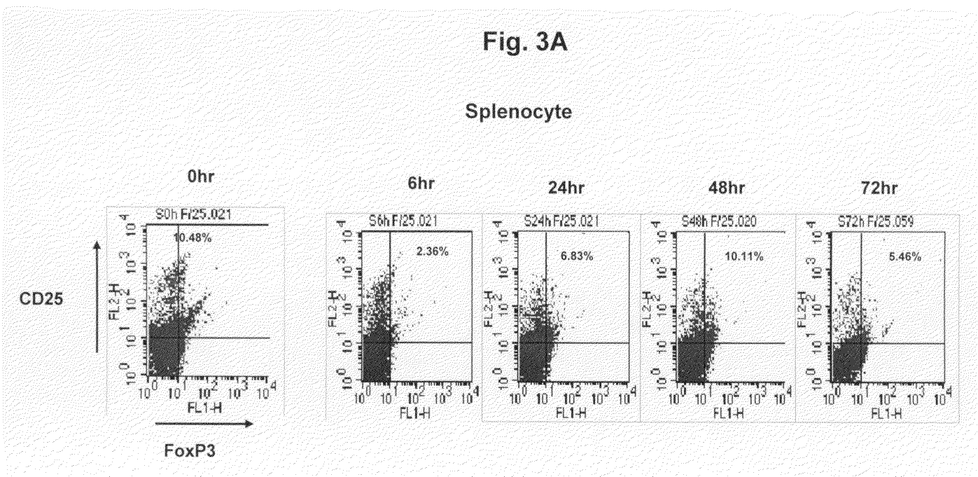

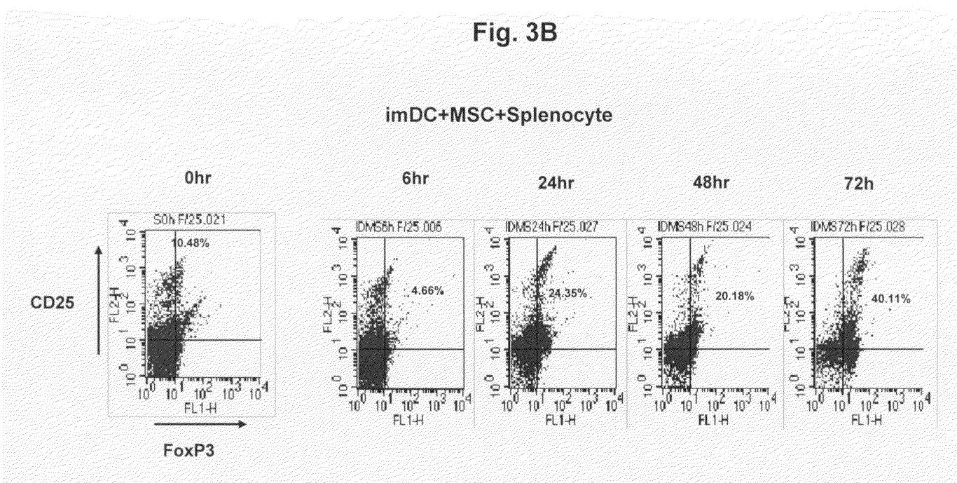

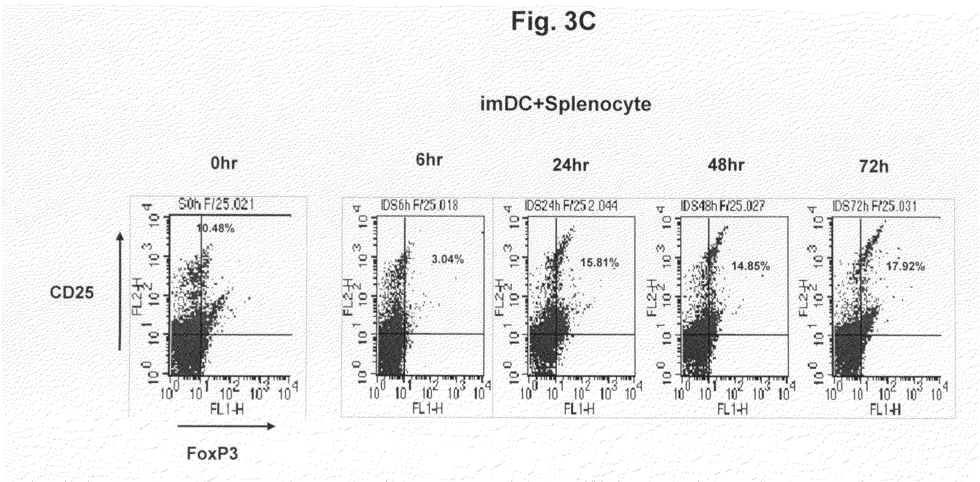

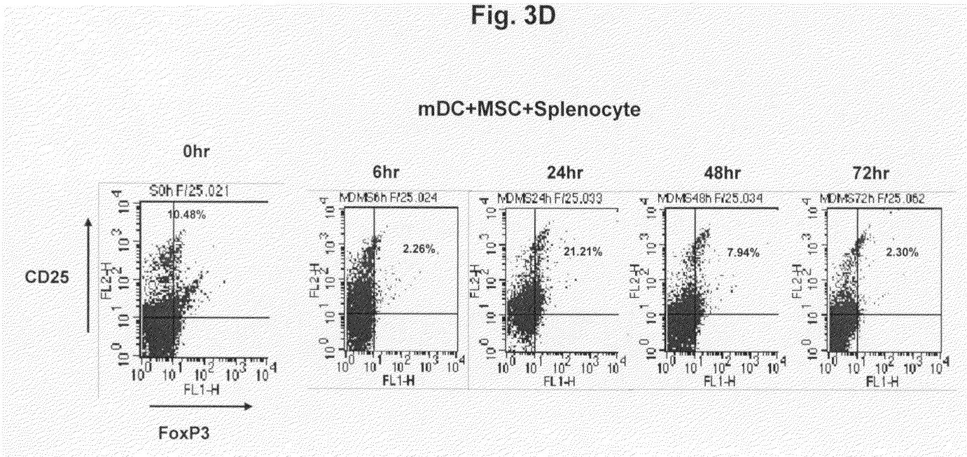

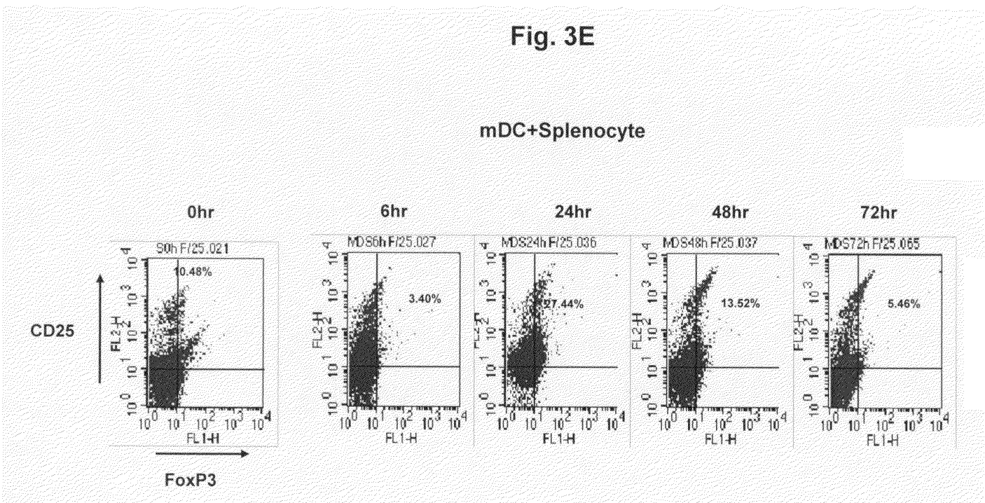

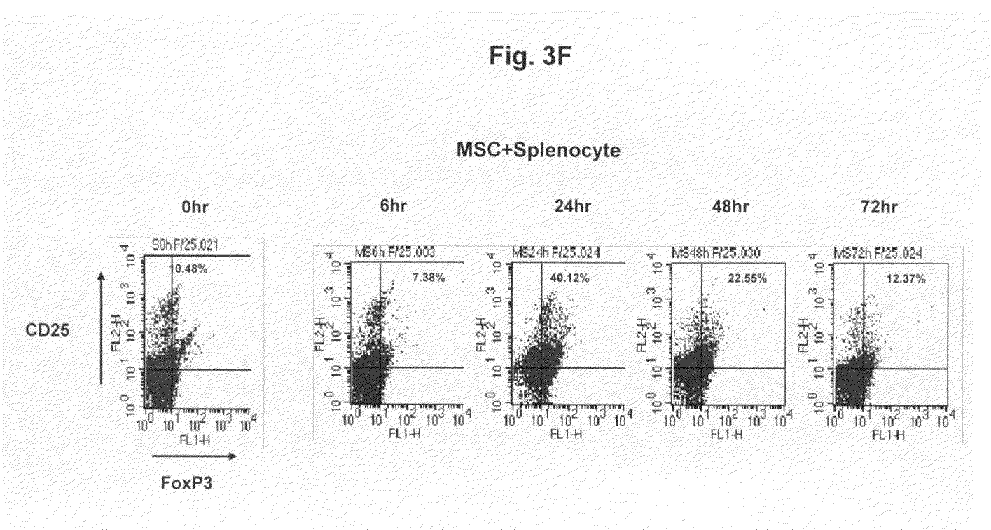

As shown in FIGS. 3A to 3F, the CD4⁺CD25⁺FoxP3⁺T_{reg} cell population was markedly induced from splenocytes mediated with MSC+imDC co-culture (40.11%, at 72 hr), as compared with that from splenocytes co-cultured with other cell combinations. The T_{reg} cell population was markedly induced from splenocytes of all test groups during the T cell-priming phase (24 hr), and thereafter the population were rapidly decreased or maintained, but dramatically increased only from splenocytes co-cultured with MSC+imDC at 72 hr after culture. Additionally, we observed that the T_{reg} cell population markedly increased from splenocytes co-cultured with MSC alone to the highest level during the T cell-priming phase, but thereafter rapidly decreased.

Consequently, these data indicated that the FoxP3⁺ T_{reg} cell population with immunosuppressive activity was prominently induced from the splenocytes co-cultured only with imDCs and MSCs over time.

MSC+imDC+Splenocyte Co-Culture Induces the Secretion of the Immunosuppressive Agent, TGF-β, in the Supernatant to a More Significant Level than imDC or MSC+Splenocyte Co-Culture

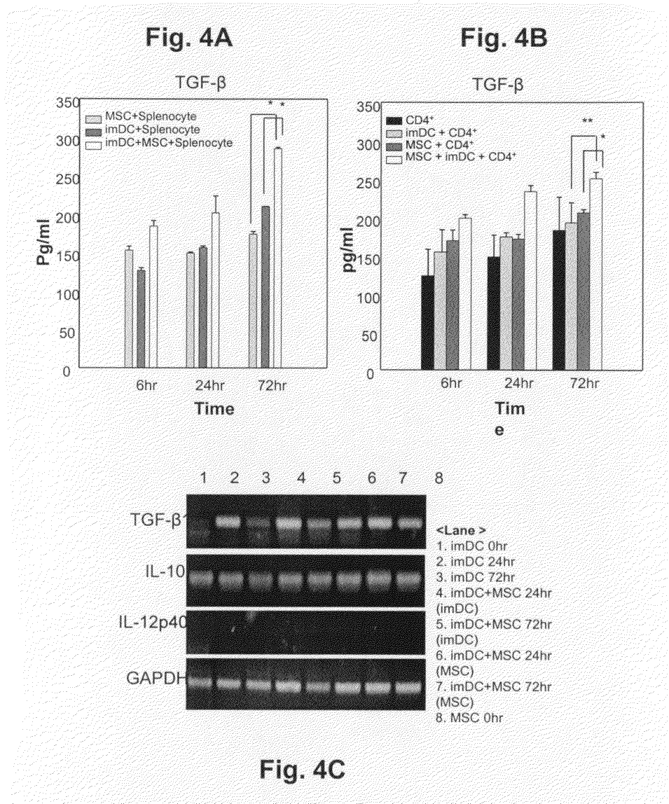

To investigate whether imDC+MSC+splenocyte or CD4 T cell co-culture could induce the secretion of the immunosuppressive agent, TGF-β, its culture supernatant was collected, and analyzed by ELISA. As shown in FIG. 4A, the TGF-β secretion was markedly induced from the imDC+MSC+splenocyte culture supernatant to a significant level (282±2.0 μg/ml) at 72 hr co-culture, compared with MSC or imDC+splenocyte co-culture (177±3.5 μg/ml and 212±0.5 μg/ml, respectively). Additionally, the co-culture experiment by use of CD4+ T cells isolated from splenocytes also showed a similar tendency to the results above (FIG. 4B). Moreover, RT-PCR analysis indicated that TGF-β transcript was highly expressed in imDCs from 72-hr imDC+MSC co-culture, compared to 72-hr imDC culture (Lane 5 and Lane 3, FIG. 4C). At 24 hr after culture, TGF-β transcript was highly expressed in both imDCs from imDC culture and imDC+MSC co-culture, but it markedly reduced in imDCs from imDC culture 72 hr after culture, while slightly lessened in imDCs from imDC+MSC co-culture. This illustrates that imDCs obviously gain a lasting immunosuppressive ability at the subcellular level, when mediated with MSCs. On the other hand, TGF-β transcript was highly expressed in MSCs from imDC+MSC co-culture even at 72 hr after culture (Lane 6 and 7). IL-10 transcript was detected to a similar level in all used cells, while IL-12 was undetectable. Together, these results suggested that imDCs could induce immunosuppressive circumstances to a further significant level at the cellular level when mediated with MSCs.

MSC+imDC+CD4 T Cell Co-Culture Attenuates the Secretion of the Th1 Cytokine, IFN-γ, in the Supernatant to a Remarkable Level, Compared to imDC+CD4 T Cell Co-Culture

In order to further investigate whether imDC+MSC+CD4 T cell co-culture could induce the secretion of Th2 cytokines or inhibit the production of Th1 cytokine, its culture supernatant was collected and analyzed by ELISA.

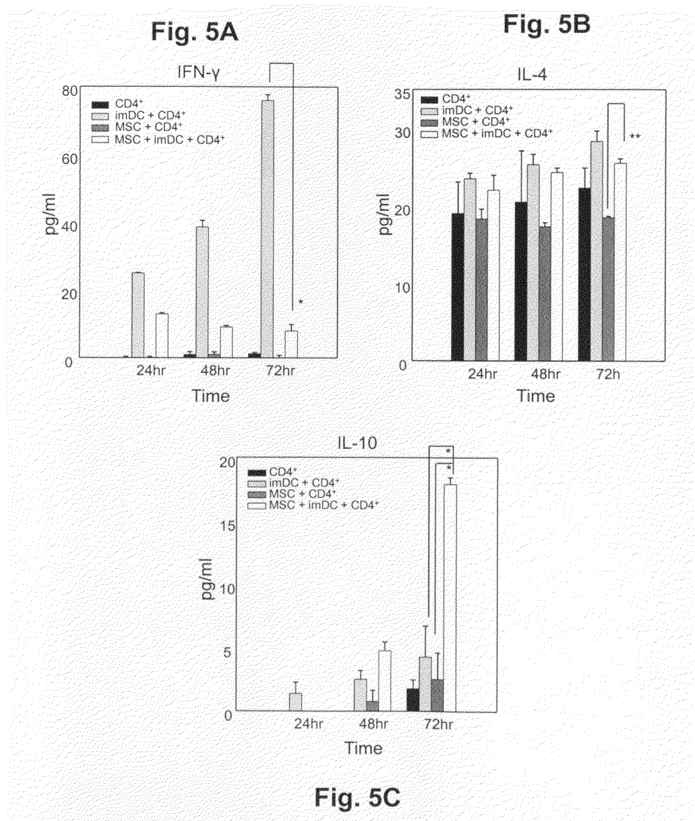

As shown in FIG. 5A, imDC+MSC+CD4 T cell co-culture dramatically inhibited (9.5±2.1 pg/ml, at 72 hr) the secretion of the Th1 cytokine, IFN-γ, which elevated by an imDC+CD4 T cell co-culture over time (77±1.9 pg/ml, at 72 hr), lowering a Th1 response. Additionally, the secretion of the Th2 cytokine, IL-4, was induced from the imDC+MSC+CD4 T cell culture supernatant to a significant level (26.5±0.5 pg/ml) at 72-hr co-culture, compared with MSC+CD4 T cell co-culture (18.5±0.2 pg/ml), but showing a slightly lower induction of the IL-4 secretion, compared to imDC+CD4 T cell co-culture (28.9±1.3 pg/ml, at 72 hr) (FIG. 5B). Moreover, imDC+MSC+CD4 T cell co-culture induced the secretion of IL-10, known to be another Th2 cytokine, to a significant level, compared to other co-culture systems, albeit showing overall lower levels (FIG. 5C).

These results indicated that pattern of Th1/Th2 cytokine production induced by imDC+MSC+CD4 T cell co-culture was distinct from that induced by imDC or MSC+CD4 T cell co-culture, presumably lowering a Th1/Th2 ratio.

The IL-10 Secretion Rate of MSC-Mediated imDCs was Increased Compared to that of imDCs.

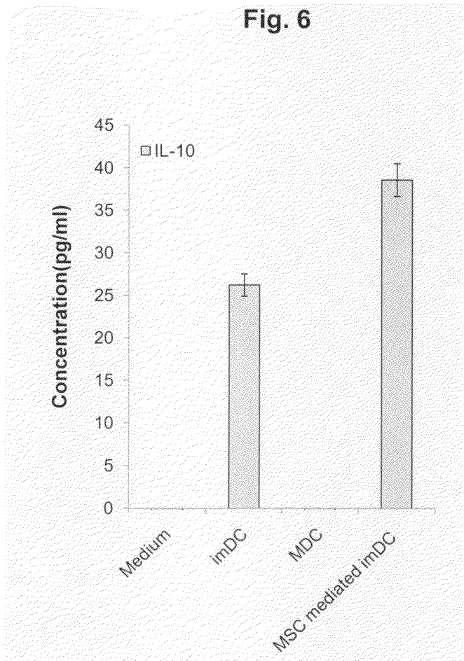

In order to investigate cytokine secreting natures of MSC-mediated imDCs, the capability of MSC-mediated imDCs to secrete IL-10 was measured. As shown in FIG. 6, mature DCs did not secrete IL-10. On the contrary, the MSC-mediated imDCs prepared according to the instant methods secreted one and half times amount of IL-10 compared to that of imDCs.

B16 Melanoma Cells are not Rejected by Balb/c Allogeneic Mice when Co-Injected with MSC-Mediated imDCs

We were also interested in examining whether tumor cells could be transplanted in MHC-mismatched allogeneic recipients by using MSC-mediated imDCs. In order to test the immunoregulatory properties of immunosuppressive cells, we implanted B16 melanoma cells in allogeneic Balb/c mice in the presence or absence of imDCs, MSCs, an imDC+MSC mix, and MSC-mediated imDCs. Particularly, to examine the systemic immunosuppressive effect, B16 melanoma cells were subcutaneously implanted at a distance of at least 2 cm instantly after immunosuppressive cell injection (subcutaneous).

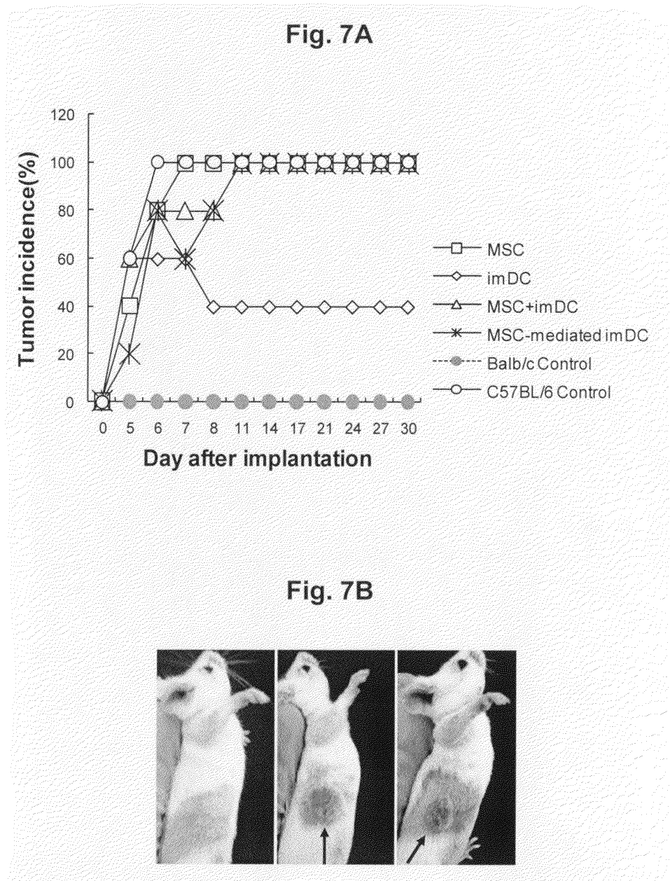

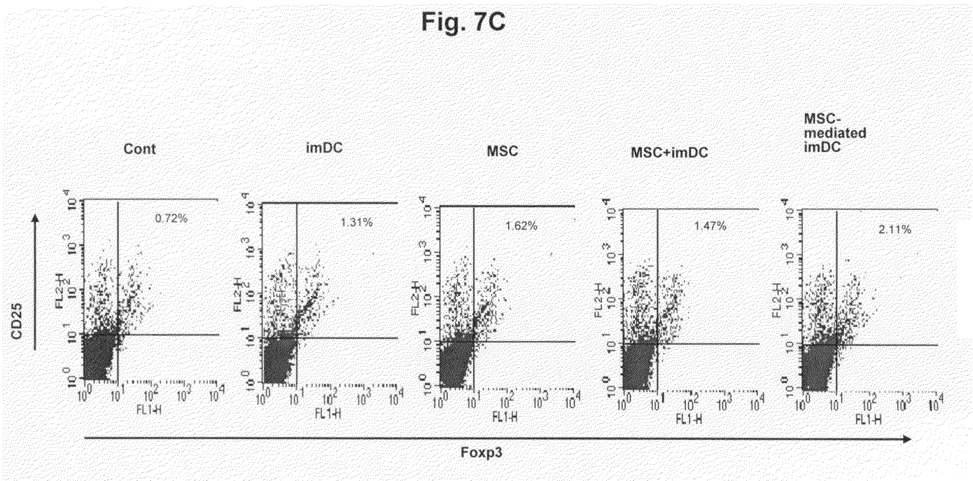

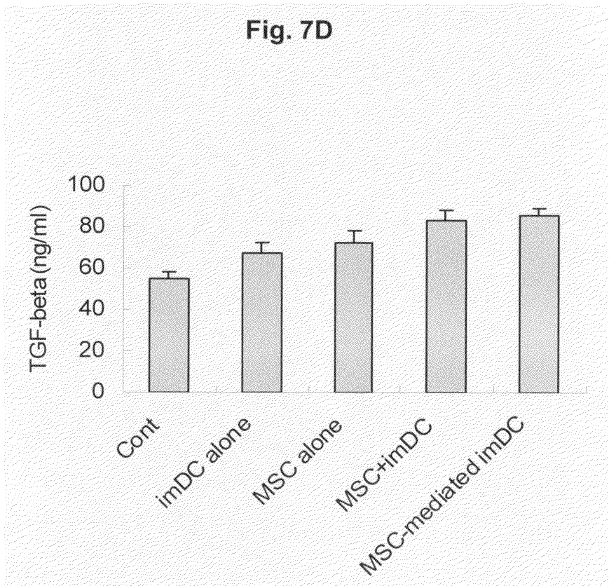

Tumor growth was compared to that of B16 cells implanted in syngeneic C57BL/6 mice (100% of tumor incidence). In all tested groups excluding the imDC-injected group, tumor incidence was 100% during the first 11 days (This tumor incidence was maintained until the mice die.) (FIG. 7a). In the control group consisting of Babl/c animals receiving only the allogeneic B16 cells, no tumor formation was observed. Photo images shown in FIG. 7b further supported the result above. The first image indicates a B16 tumor-injected Balb/c mouse, being not given the immunosuppressive cells, and the second and third images were photographed with different individuals in the MSC-mediated imDC group.

Data on in vivo immune status were in line with the results above (FIGS. 7c and 7d). We confirmed that the CD25⁺ Foxp3⁺ T_{reg} cell population in CD4 T cell isolated from the spleen of immunosuppressed tumor-bearing mice increased 2˜3 times more than that of mice in the Balb/c control group (consisting of B16 tumor-injected Balb/c mice untreated with the immunosuppressive cells) (FIG. 7c).

Additionally, the systemic TGF-β concentration was found in the sera of immunosuppressed tumor-bearing mice to a higher level than in those of only B16 cell-injected mice (FIG. 7d). Taken together, these results suggested that MSC-mediated imDCs induced a potent immunosuppressive effect at least along with an increase of the Foxp3 specific-T_{reg} cell population, being similar to that of MSCs.

Survival Rate of Balb/c Mice Grafted with B16 Melanoma Cells is Increased when Co-Injected with MSC-Mediated imDCs

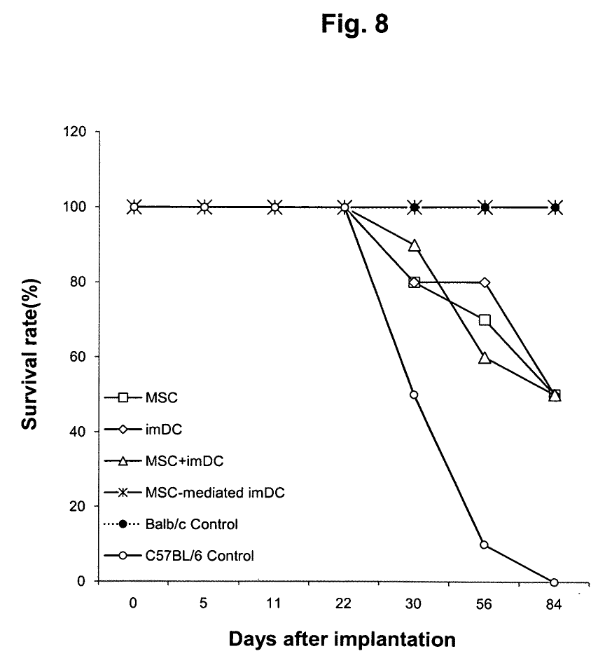

In addition to the capability of MSC-mediated imDCs to reduce the tumor graft rejection, it was also demonstrated that MSC-mediated imDCs of the present invention could increase the survival rate of Balb/c mice grafted with B16 melanoma cells. The Balb/c mice were injected with MSCs, imDCs, imDCs+MSCs, or MSC-mediated imDCs respectively and after that subcutaneously grafted with B16 melanoma cells. Afterwards, the survival rates of Balb/c mice were determined. As shown in FIG. 8, when MSCs-mediated imDCs were injected, the survival rate of Balb/c mice grafted with B16 melanoma cells was increased about two times compared to the injection of other immune cells of MSCs, imDCs, or imDCs+MSCs (simple mixture of imDCs and MSCS).

Having described a preferred embodiment of the present subject matter, it is to be understood that variants and modifications thereof falling within the spirit of the subject matter may become apparent to those skilled in the art, and the scope of this subject matter is to be determined by the appended claims and their equivalents.

### REFERENCE

1. Pittenger, M. F., A. M. Mackay, S. C. Beck, R. K. Jaiswal, R. Douglas, J. D. Mosca, M. A. Moorman, D. W. Simonetti, S. Craig, and D. R. Marshak. 1999. Multilineage potential of adult human mesenchymal stem cells. Science 284:143-147.
2. Pereira, R. F., K. W. Halford, M. D. O'Hara, D. B. Leeper, B. R Sokolov, M. D. Pollard, O. Bagasra, and D. J. Prockop. 1995. Cultured adherent cells from marrow can serve as long-lasting precursor cells for bone, cartilage, and lung in irradiated mice. Proc Natl Acad Sci USA 92:4857-4861.
3. Wakitani, S., T. Saito, and A. I. Caplan. 1995. Myogenic cells derived from rat bone marrow mesenchymal stem cells exposed to 5-azacytidine. Muscle Nerve 18:1417-1426.
4. Kadiyala, S., R. G. Young, M. A. Thiede, and S. P Bruder. 1997. Culture expanded canine mesenchymal stem cells possess osteochondrogenic potential in vivo and in vitro. Cell Transplant 6:125-134.
5. Mosca, J. D. J. K. Hendricks, D. Buyaner, J. Davis-Sproul, L. C. Chuang, M. K. Majumdar, R. Chopra, F. Barry, M. Murphy, M. A. Thiede, U. Junker, R. J. Rigg, S. P. Forestell, E. Bohnlein, R. Storb, and B. M. Sandmaier. 2000. Mesenchymal stem cells as vehicles for gene delivery. Clin Orthop Relat Res:S71-90.
6. Martin, D. R., N. R. Cox, T. L. Hathcock, G. P Niemeyer, and H. J. Baker. 2002. Isolation and characterization of multipotential mesenchymal stem cells from feline bone marrow. Exp Hematol 30:879-886.
7. Phinney, D. G., G. Kopen, R. L. Isaacson, and D. J. Prockop. 1999. Plastic adherent stromal cells from the bone marrow of commonly used strains of inbred mice: variations in yield, growth, and differentiation. J Cell Biochem 72:570-585.
8. Bartholomew, A., C. Sturgeon, M. Siatskas, K. Ferrer, K. McIntosh, S. Patil, W. Hardy, S. Devine, D. Ucker, R. Deans, A. Moseley, and R. Hoffman. 2002. Mesenchymal stem cells suppress lymphocyte proliferation in vitro and prolong skin graft survival in vivo. Exp Hematol 30:42-48.
9. Di Nicola, M., C. Carlo-Stella, M. Magni, M. Milanesi, P. D. Longoni, P. Matteucci, S. Grisanti, and A. M. Gianni. 2002. Human bone marrow stromal cells suppress T-lymphocyte proliferation induced by cellular or nonspecific mitogenic stimuli. Blood 99:3838-3843.
10. Ringden, O., M. Labopin, A. Bacigalupo, W. Arcese, U. W. Schaefer, R. Willemze, H. Koc, D. Bunjes, E. Gluckman, V. Rocha, A. Schattenberg, and F. Frassoni. 2002. Transplantation of peripheral blood stem cells as compared with bone marrow from HLA-identical siblings in adult patients with acute myeloid leukemia and acute lymphoblastic leukemia. J Clin Oncol 20:4655-4664.
11. Jonuleit, H., E. Schmitt, K. Steinbrink, and A. H. Enk. 2001. Dendritic cells as a tool to induce anergic and regulatory T cells. Trends Immunol 22:394-400.
12. Steinman, R. M., and M. C. Nussenzweig. 2002. Avoiding horror autotoxicus: the importance of dendritic cells in peripheral T cell tolerance. Proc Natl Acad Sci USA 99:351-358.
13. Vosters, O., J. Neve, D. De Wit, F. Willems, M. Goldman, and V. Verhasselt. 2003. Dendritic cells exposed to nacystelyn are refractory to maturation and promote the emergence of alloreactive regulatory t cells. Transplantation 75:383-389.
14. Mahnke, K., E. Schmitt, L. Bonifaz, A. H. Enk, and H. Jonuleit. 2002. Immature, but not inactive: the tolerogenic function of immature dendritic cells. Immunol Cell Biol 80:477-483.
15. Steinman, R. M., D. Hawiger, and M. C. Nussenzweig. 2003. Tolerogenic dendritic cells. Annu Rev Immunol 21:685-711.
16. Roncarolo, M. G., M. K. Levings, and C. Traversari. 2001. Differentiation of T regulatory cells by immature dendritic cells. J Exp Med 193:F5-9.
17. de Heusch, M., G. Oldenhove, J. Urbain, K. Thielemans, C. Maliszewski, O. Leo, and M. Moser. 2004. Depending on their maturation state, splenic dendritic cells induce the differentiation of CD4(+) T lymphocytes into memory and/or effector cells in vivo. Eur J Immunol 34:1861-1869.
18. Lutz, M. B., and G. Schuler. 2002. Immature, semi-mature and fully mature dendritic cells: which signals induce tolerance or immunity. Trends Immunol 23:445-449.
19. Rutella, S., S. Danese, and G. Leone. 2006. Tolerogenic dendritic cells: cytokine modulation comes of age. Blood 108:1435-1440.
20. Yamazaki, S., T. Iyoda, K. Tarbell, K. Olson, K. Velinzon, K. Inaba, and R. M. Steinman. 2003. Direct expansion of functional CD25⁺ CD4⁺ regulatory T cells by antigen-processing dendritic cells. J Exp Med 198:235-247.
21. Pasare, C., and R. Medzhitov. 2003. Toll pathway-dependent blockade of CD4⁺ CD25⁺ T cell-mediated suppression by dendritic cells. Science 299:1033-1036.
22. Martin, E., B. O'Sullivan, P. Low, and R. Thomas. 2003. Antigen-specific suppression of a primed immune response by dendritic cells mediated by regulatory T cells secreting interleukin-10. Immunity 18:155-167.
23. Romani N, Gruner S, Brang D, et al. Proliferating dendritic cell progenitors in human blood. J Exp Med. 1994; 180:83-93.
24. Deans R J, Moseley A B. Mesenchymal stem cells; biology and potential clinical uses. Exp Hematol. 2000; 28:875-884.
25. Sakaguchi, S. 2005. Naturally arising Foxp3-expressing CD25⁺ CD4⁺ regulatory T cells in immunological tolerance to self and non-self. Nat. Immunol. 6:345-352.
26. von Boehmer, H. 2005. Mechanisms of suppression by suppressor T cells. Nat. Immunol. 6:338-344.
27. Ghiringhelli, F., C. Menard, M. Terme, C. Flament, J. Taieb, N. Chaput, P. E. Puig, S. Novault, B. Escudier, E. Vivier, et al. 2005. CD4⁺ CD25⁺ regulatory T cells inhibit natural killer cell functions in a transforming growth factor-beta-dependent manner. J. Exp. Med. 202:1075-1085.
28. Meirelles Lda, S., and N. B. Nardi. 2003. Murine marrow-derived mesenchymal stem cell: isolation, in vitro expansion, and characterization. Br J Haematol 123:702-711.
29. Sun, S., Z. Guo, X. Xiao, B. Liu, X. Liu, P. H. Tang, and N. Mao. 2003. Isolation of mouse marrow mesenchymal progenitors by a novel and reliable method. Stem Cells 21:527-535.
30. Read, S., S. Mauze, C. Asseman, A. Bean, R. Coffman, and F. Powrie. 1998. CD38+ CD45RB(low) CD4+ T cells: a population of T cells with immune regulatory activities in vitro. Eur J Immunol 28:3435-3447.
31. Thornton, A. M., and E. M. Shevach. 2000. Suppressor effector function of CD4⁺ CD25⁺ immunoregulatory T cells is antigen nonspecific. J. Immunol 164:183-190.

1. A mesenchymal stem cell-mediated dendritic cell for suppressing immune responses.

2. The mesenchymal stem cell-mediated dendritic cell according to claim 1, wherein the dendritic cell is autologous.

3. The mesenchymal stem cell-mediated dendritic cell according to claim 1, wherein the dendritic cell, after co-cultured with mesenchymal stem cells, has an enhanced potential to suppress T cell immunity or an enhanced potential to induce regulatory T cells.

4. The mesenchymal stem cell-mediated dendritic cell according to claim 1, wherein the dendritic cell, after co-cultured with mesenchymal stem cells, has a potential to suppress the secretion of inflammatory cytokines and to promote the secretion of immunosuppressive cytokines.

5. The mesenchymal stem cell-mediated dendritic cell according to claim 1, wherein the dendritic cell, after co-cultured with mesenchymal stem cells, has reduced CD86 expression level compared to the dendritic cells untreated with mesenchymal stem cells.

6. The mesenchymal stem cell-mediated dendritic cell according to claim 1, wherein the dendritic cell, after co-cultured with mesenchymal stem cells, has increased CD80 expression level compared to the dendritic cells untreated with mesenchymal stem cells.

7. The mesenchymal stem cell-mediated dendritic cell according to claim 1, wherein the dendritic cell, after co-cultured with mesenchymal stem cells, has an increased potential to secrete IL (interleukin)-10 compared to the dendritic cells untreated with mesenchymal stem cells.

8. A method for preparing dendritic cells, which comprises the steps of:
(a) preparing dendritic cells;
(b) preparing mesenchymal stem cells;
(c) co-culturing the dendritic cells with the mesenchymal stem cells; and
(d) isolating dendritic cells having an enhanced potential to suppress immune responses from the co-cultured medium.

9. The method according to claim 8, wherein the dendritic cells in the step (a) are autologous.

10. The method according to claim 8, wherein the dendritic cells in the step (a) are immature cells.

11. The method according to claim 8, wherein the mesenchymal stem cells are syngeneic, allogeneic or xenogeneic to the dendritic cells.

12. The method according to claim 8, wherein the co-culturing is carried out for 0.1-200 hr.

13. The method according to claim 8, wherein the dendritic cells in the step (d) have reduced CD86 expression level compared to the dendritic cells in the step (a).

14. The method according to claim 8, wherein the dendritic cells in the step (d) have increased CD80 expression level compared to the dendritic cells in the step (a).

15. A method for suppressing immune responses, which comprises administering to a subject in need for immune suppression a pharmaceutically effective amount of mesenchymal stem cells-mediated immature dendritic cells.

16. The method according to claim 15, wherein the mesenchymal stem cells-mediated immature dendritic cells are autologous.

17. The method according to claim 15, wherein the mesenchymal stem cells-mediated immature dendritic cells have reduced CD86 expression level compared to the dendritic cells untreated with mesenchymal stem cells.

18. The method according to claim 15, wherein the mesenchymal stem cells-mediated immature dendritic cells have increased CD80 expression level compared to the dendritic cells untreated with mesenchymal stem cells.

19. The method according to claim 15, wherein the mesenchymal stem cells-mediated immature dendritic cells have an increased potential to secrete IL (interleukin)-10 compared to the dendritic cells untreated with mesenchymal stem cells.

20. The method according to claim 15, wherein the mesenchymal stem cells-mediated immature dendritic cells have a potential to increase the population of CD25⁺ Foxp3⁺ T_{reg} cells.

21. The method according to claim 15, wherein the subject suffers from tissue or organ transplantation rejection, autoimmune disease, or inflammatory disease.

22. The method according to claim 21, wherein the autoimmune disease is rheumatoid arthritis, diabetics, or atopic dermatitis.

23. The method according to claim 15, wherein the mesenchymal stem cells-mediated immature dendritic cells are prepared according to the method comprising the steps of:
(a) preparing immature dendritic cells;
(b) preparing mesenchymal stem cells;
(c) co-culturing the immature dendritic cells with the mesenchymal stem cells; and
(d) isolating immature dendritic cells having an enhanced potential to suppress immune responses from the co-cultured medium.
